(19) United States
(12) Patent Application Publication
(10) Pub. No.: US 2008/0008754 A1
(43) Pub. Date: Jan. 10, 2008

(54) Pharmaceutical Compositions Comprising (+)-(2S,3S)-2-(3-Chlorophenyl)-3,5,5-Trimethyl-2-Morpholinol

(75) Inventors: Karen Jane Lewis, Cidra, PR (US); Ngoc-Anh Thi Nguyen, Durham, NC (US); Damiano Papini, Verona (IT); Francesco Roberto, Verona (IT); Mickey Lee Wells, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: SMITHKLINE BEECHAM CORPORATION, PHILADELPHIA, PA (US)

(21) Appl. No.: 11/719,409

(22) PCT Filed: Nov. 16, 2005
(86) PCT No.: PCT/EP05/12368

§ 371 Date: May 16, 2007

(30) Foreign Application Priority Data
Nov. 18, 2004 (GB) 0425445.4

(51) Int. Cl. A61K 9/36; A61K 31/535; A61P 25/22; A61P 25/24; A61P 3/04; A61P 43/00

(52) U.S. Cl. 424/465; 514/239.5

(57) ABSTRACT

Novel pharmaceutical compositions, particularly sustained release pharmaceutical compositions, of (+)-(2S,3S)-2-(3-chlorophenyl)-3,5,5-trimethyl-2-morpholinol or pharmaceutically acceptable salts or solvates thereof are disclosed.

The present invention relates to novel pharmaceutical compositions, particularly sustained release pharmaceutical compositions, of (+)-(2S,3S)-2-(3-chlorophenyl)-3,5,5-trimethyl-2-morpholinol (hereinafter the “compound of formula (I)”) or pharmaceutically acceptable salts or solvates thereof.

### BACKGROUND OF THE INVENTION

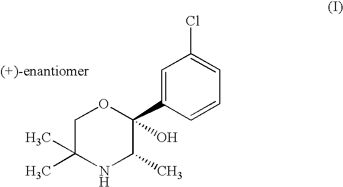

The compound of formula (I) and its salts and solvates have been disclosed as being of use in the treatment of depression (including major depressive disorder (MDD), bipolar depression (type I and II), major (unipolar) depression and depression with atypical features (e.g. lethargy, over-eating/obesity, hypersomnia)), attention deficit hyperactivity disorder (ADHD), obesity, migraine, pain (including neuropathic pain, e.g. diabetic neuropathy, sciatica, non-specific lower back pain, multiple sclerosis pain, fibromyalgia, HIV-related neuropathy, neuralgia such as post-herpetic neuralgia and trigeminal neuralgia and pain resulting from physical trauma, amputation, cancer, toxins or chronic inflammatory conditions), sexual dysfunction (including inhibited sexual desire (low libido), inhibited sexual arousal or excitement, orgasm dysfunction, inhibited female orgasm, inhibited male orgasm, hypoactive sexual desire disorder (HSDD), female sexual desire disorder (FSDD) and sexual dysfunction side-effects induced by treatment with antidepressants of the SSRI-class), Parkinson's disease (including relief from the symptoms of Parkinson's disease which include, but are not limited to, locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), Alzheimer's disease, or addiction to cocaine or nicotine-containing (especially tobacco) products (WO 99/37305 and US2003-0064988; both Glaxo Group Limited).

US2003-0032643 (Glaxo Group Limited) discloses the use of the compound of formula (I) and its salts and solvates in the treatment of seasonal affective disorder, chronic fatigue, narcolepsy and cognitive impairment.

US2003-0083330 (Glaxo Group Limited) discloses the use of the compound of formula (I) and its salts and solvates in the treatment of addiction to alcohol.

WO 00/51546 and WO 01/62257 (both Sepracor Inc) disclose the use of a bupropion metabolite in the treatment of a disorder that is ameliorated by the inhibition of neuronal monoamine reuptake, sexual dysfunction (including erectile dysfunction), an affective disorder (including depression, anxiety disorders, attention deficit hyperactivity disorder, bipolar and manic conditions, sexual dysfunction, psycho-sexual dysfunction, bulimia, obesity or weight gain, narcolepsy, chronic fatigue syndrome, seasonal affective disorder, premenstrual syndrome, and substance addiction or abuse), nicotine addiction, a cerebral function disorder (including senile dementia, Alzheimer's type dementia, memory loss, amnesia/amnestic syndrome, epilepsy, disturbances or consciousness, coma, lowering of attention, speech disorders, Parkinson's disease, Lennox syndrome, autistic disorder, autism, hyperkinetic syndrome, schizophrenia, cerebral infarction, cerebral bleeding, cerebral auteriosclerosis, cerebral venous thrombosis and head injury), epilepsy, smoking cessation and incontinence.

WO 2005/051395 (SmithKline Beecham Corporation) discloses the use of the compound of formula (I) and its salts and solvates in the treatment of anxiety disorders or in the treatment of mixed anxiety-depressive disorder.

WO 2005/053700 (SmithKline Beecham Corporation) discloses the use of the compound of formula (I) and its salts and solvates in the treatment of restless legs syndrome (RLS) or in the treatment of periodic limb movement disorder (PLMD).

### SUMMARY OF THE INVENTION

An object of the present invention is to provide a pharmaceutical composition of the compound of formula (I) or a pharmaceutically acceptable salt or solvate thereof, which composition is adapted to provide an effective therapeutic dosage of the compound of formula (I) to a human subject by means of once-daily oral administration.

Thus in an aspect of the present invention there is provided a sustained release pharmaceutical composition comprising the compound of formula (I) or a pharmaceutically acceptable salt or solvate thereof.

In another aspect of the present invention there is provided a sustained release pharmaceutical composition for oral administration to a human subject comprising the compound of formula (I) or a pharmaceutically acceptable salt or solvate thereof.

In another aspect of the present invention there is provided a sustained release pharmaceutical composition, which composition is adapted to provide an effective therapeutic dosage of the compound of formula (I) to a human subject by means of once-daily oral administration.

In another aspect there is provided a method of treatment of the disorders and conditions referred to above in a human subject, and also including the treatment of anxiety disorders and mixed depressive-anxiety disorder, comprising oral administration to said subject of a sustained release pharmaceutical composition as referred to above.

In another aspect there is provided the use of a sustained pharmaceutical composition as referred to above in the manufacture of a medicament for the treatment of the disorders and conditions in a human subject as referred to above.

### DETAILED DESCRIPTION OF THE INVENTION

As used herein the term ‘pharmaceutically acceptable’ embraces both human and veterinary use: for example the term ‘pharmaceutically acceptable’ embraces a veterinarily acceptable compound.

The compound of formula (I) or a salt or solvate thereof is included within the compositions of the present invention in an enantiomerically pure form.

As used herein, “enantiomerically pure” means that the composition contains greater than about 95% of the (2S,3S) enantiomer by weight, more preferably greater than about 99% of the (2S,3S) enantiomer by weight, most preferably greater than 99.5% of the (2S,3S) enantiomer by weight, said weight percent based upon the total weight of the compound of formula (I) and all diastereomers thereof.

Suitable for use in pharmaceutical compositions according to the present invention are pharmaceutically acceptable salts or solvates of the compound of formula (I), particularly those disclosed in U.S. Pat. No. 6,342,496 B1, U.S. Pat. No. 6,337,328 B1, U.S. Pat. No. 6,391,875 B1, U.S. Pat. No. 6,274,579 B1, U.S. Patent Application Publication Nos. 2002/0052340 A1, 2002/0052341 A1, and 2003/0027827 A1, as well as WO 01/62257, WO 99/37305, WO 00/51546 and WO 01/62257. Suitable pharmaceutically acceptable salts can include, but are not limited to, hydrochloride salt, hydrogen sulphate salt and other sulphate salts, hydrogen phosphate salt and other phosphate salts, methanesulfonate salt, p-toluenesulfonate salt, citrate salt, fumarate salt, tartrate salt, and the like. Of these, (+)-(2S,3S)-2-(3-chlorophenyl)-3,5,5-trimethyl-2-morpholinol hydrochloride is particularly preferred.

The compound of formula (I) or a salt or solvate thereof may be prepared in isolated form, and preferably in an enantiomerically pure form, in accordance with the procedures set forth in WO 99/37305, US2003-0064988, US2003-0032643 and US2003-0027827 (all of Glaxo Group Limited), in accordance with the procedures set forth in WO 00/51546 and WO 01/62257 (both of Sepracor Inc.), or in accordance with the procedures set forth in WO 2005/040141 and WO 2005/040140 (both of SmithKline Beecham Corporation).

In an embodiment of the present invention is a method or a use of the pharmaceutical composition of the present invention as defined above wherein the condition or disorder being treated is selected from depression, pain, chronic fatigue, obesity, anxiety disorders and mixed depressive-anxiety disorder.

In an embodiment of the present invention is a method or a use of the pharmaceutical composition of the present invention as defined above wherein the condition or disorder being treated is depression.

In an embodiment of the present invention is a method or a use of the pharmaceutical composition of the present invention as defined above wherein the condition or disorder being treated is pain.

In an embodiment of the present invention is a method or a use of the pharmaceutical composition of the present invention as defined above wherein the condition or disorder being treated is obesity.

The amount of the compound of formula (I) or a salt or solvate thereof required to achieve the desired therapeutic effect (“an effective therapeutic dosage”) will, of course depend on a number of factors, for example, the particular disorder or condition being treated, the mode of administration and the recipient being treated. In general, the daily dose (given as a single once-daily dose) will be in the range of about 0.15 to about 1.2 mg/kg, or about 0.15 to 2.4 mg/kg, or about 0.3 to 2.5 mg/kg. In certain circumstances as directed by a physician, the dosage may be given as divided doses throughout the day.

The pharmaceutical composition of the compound of formula (I) or a pharmaceutically acceptable salt or solvate thereof will comprise one or more pharmaceutically acceptable carriers, diluents or excipients. The carriers, diluents and excipients must, of course, be acceptable in the sense of being compatible with the other ingredients of the formulation and must not be deleterious to the recipient. The carrier is formulated with the active ingredient as an orally administered unit-dose formulation, for example a tablet containing 10 mg, 20 mg, 40 mg, 60 mg, 80 mg, 100 mg, 120 mg, 140 mg, 150 mg, or 160 mg of the compound of formula (I) or a salt or solvate thereof. In embodiments of the present invention, the composition contains 10-80 mg, or 20-100 mg, or 40-120 mg, or 60-140 mg, or 80-140 mg, or 80-160 mg, or 100-160 mg (all expressed as the weight of the free base) of the compound of formula (I) or a pharmaceutically acceptable salt or solvate thereof. In another embodiment the composition contains 180-240 mg (expressed as the weight of the free base) of the compound of formula (I) or a pharmaceutically acceptable salt or solvate thereof.

Solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice.

Compositions may, if desired, be in the form of a pack accompanied by written or printed instructions for use.

Suitable compositions for use in the present invention thus include sustained release solid-dosage formulations, optionally film-coated solid-dosage formulations, and especially tablet and caplet formulations, for oral once-daily administration of the compound of formula (I).

Sustained release is typically provided by use of a sustained release matrix, usually in tablet form, such as disintegrating, non-disintegrating or eroding matrices. Alternative methods of achieving sustained release are well-known to the person skilled in the art and include diffusion-core, bead formulations or barrier-coated tablets.

Scheme 1 illustrates the presumed degradation of the compound of formula (I) in aqueous solution (stored at room temperature for 3-4 weeks):

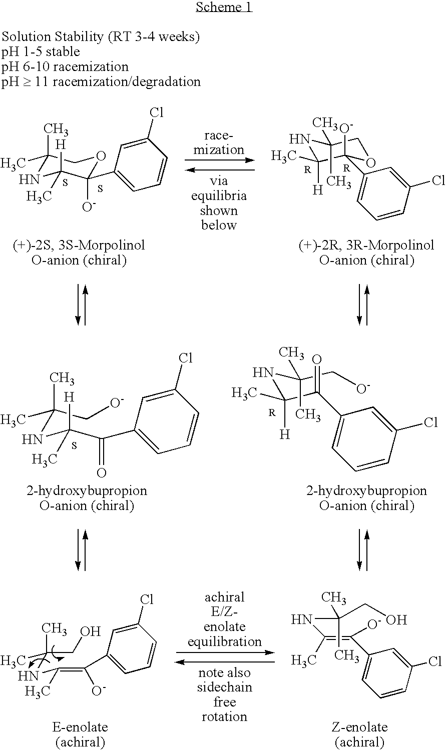

The degradation pathway of the compound of formula (I) is thought to proceed via morpholinol ring opening, which also leads to chiral inversion forming the (2R,3R)-enantiomer of the compound of formula (I) and ultimately racemization. The compound of formula (I) as a drug substance alone is relatively stable, but degradation may result following formulation with conventional pharmaceutical excipients, heat or humidity.

It has also been found that the formation of the (2R,3R)-enantiomer and degradation products may be reduced in an acidic environment and thus to maintain the physical and chemical stability of the sustained release pharmaceutical compositions of the present invention they suitably contain an acidic stabiliser.

The precise nature and amount of the acidic stabiliser needs to be such that formation of the (2R,3R) enantiomer is prevented and so that the stabiliser does not have an adverse effect (for example degradation) on any rate-controlling polymers present. For example, too little acidic stabiliser, or a local high pH environment, may not provide sufficient protection against formation of the (2R,3R) enantiomer, whereas too much acidic stabiliser, or a local low pH environment, may protect against formation of the (2R,3R) enantiomer, but may have the potential to cause degradation of any rate-controlling polymer present, either immediately or over a period of time. Degradation of any rate-controlling polymer present may result in a loss of function, and therefore increased release rate of the active ingredient and potentially loss of control and dose dumping.

Suitably the acidic stabiliser will be stable in itself, non volatile and compatible with the active drug substance and excipients, and will typically provide a suitable acidic micro-environment. Suitable acidic stabilisers include citric acid, L-cysteine HCl, glycine HCl, hydrochloric acid, malic acid, nitric acid, phosphoric acid, sodium metabisulphite, sulphuric acid, tartaric acid, alginic acid, ethane disulfonic, 1,2-ethylene diamine dihydrochloride, and isethionic acid, to be used alone or in combination.

Although the above-mentioned acid stabilisers are all suitable for use in stabilising a sustained release formulation of the compound of formula (I), by virtue of creating an acidic environment typically in the pH range of 2.5-3.5 (for a slurry of 1 tablet into 5 ml of water), there are potential disadvantages in their inclusion in a pharmaceutical formulation, due to their reactivity, volatility, and/or safety. Disadvantages of their use may include equipment corrosion during manufacture, safety during handling and storage of these materials, odour, colour discoloration, and formation of adducts within the formulation.

It has been found that, surprisingly, sodium bisulphate, the monosodium salt of sulphuric acid (also known as sodium hydrogen sulphate and sodium acid sulphate), provides an enhanced stability of the compound of formula (I) in sustained release formulations at equivalent hydrogen ion concentrations to the above acids.

The use of sodium bisulphate as an acidic stabiliser in a matrix formulation resulted in 50% less total impurities and 46% less (2R,3R) enantiomer than the Normal (or acid) equivalent amount of sulphuric acid, as shown in Table 1 below. In addition the use of sodium bisulphate has the following significant advantages in that it is a solid (which minimises handling hazards), it is non-corrosive, and non-volatile, with reduced reactivity compared to other acids listed which also have an anti-oxidant activity. Sodium bisulphate is commercially available as an anhydrous form (NaHSO₄) or as a monohydrate (NaHSO₄.H₂O), either of which may be used.

TABLE 1
Comparison of sulphuric acid and sodium bisulphate on
the total level of impurities and (2R,3R)-enantiomer

| | | | Total | (2R,3R)- |
|---|---|---|---|---|
| | Storage | | Impurities | enantiomer |
| Stabiliser | Condition | Time | % a/a | % a/a |
| Sulphuric Acid | 40° C./75% RH | 6 months | 0.35 | 0.48 |
| 0.3% w/w | | | | |
| Sodium | 40° C./75% RH | 6 months | 0.17 | 0.27 |
| Bisulphate | | | | |
| 0.8% w/w | | | | |

% a/a represents the percentage of the peak area compared to the peak area for the active in a chromatographic trace

Potassium bisulphate is an alternative acidic stabiliser which is expected to also provide similar advantages. The amounts of potassium bisulphate used in sustained released formulations to achieve such stabilisation would typically be the same as the amounts of sodium bisulphate referred to below.

Tablets and capsules for oral administration are usually presented in a unit dose, and contain conventional excipients such as binding agents, fillers, diluents, tabletting agents (compression aids), lubricants, disintegrants, colorants, flavourings, and wetting agents.

Matrix Formulations

A typical matrix formulation is a tablet, for example an aqueous film-coated tablet, comprising of a single layer. Typically the tablet may be a round tablet between about 5 mm and 15 mm diameter or a capsule-shaped tablet about 12 to 17 mm×5 to 7.7 mm.

Preparation process: The drug substance is generally blended and wet granulated with pharmaceutically acceptable excipients, including a rate-controlling polymer. An acidic stabiliser is added directly to the other granulation excipients (e.g. alginic acid) or is first dissolved in purified water (e.g. sodium bisulphate, sulphuric acid) to produce the granulation solution, and the granule is produced by conventional processing techniques, for example either a high shear or a fluid bed process, followed by drying, milling, blending, compression and optionally coating. Tablets may be coated according to well known methods in the art. The release rate can be further controlled by changes to the polymer grade and level, excipient type and level, and the incorporation of multiple layers with different release rates. A change in dose can be produced by increasing the level of drug substance, and decreasing the level of excipients, while adjusting the level of rate-controlling polymer accordingly. Alternatively the compression blend can be compressed with an increased or decreased weight as appropriate whilst maintaining a similar surface area to volume ratio.

Suitable rate-controlling polymers may be pH-dependent or pH-independent and include:—aliphatic polyesters (homo and co-polymers of polylactic polyglycolic, polyhydroxybutyrate, polyvaleric and polycaprolactone), carbomers, carnauba wax, carrageenan, carboxymethylcellulose sodium, cellulose acetate, cellulose acetate butyrate, cellulose acetate phthalate, cellulose acetate trimellitate (CAT), emulsifying wax, ethylcellulose, glycerol monostearate, glycerol palmitostearate, glyceryl behenate, guar gum, hydrogenated castor oil, hydroxyethylcellulose, hydroxypropylmethylcellulose, hydroxypropylmethylcellulose acetate succinate, methylcellulose, hydroxypropylmethylcellulose phthalate, polyethylene glycol, polyamides, polyethylene oxide, polymethacrylates, polyvinyl acetate phthalate, sodium alginate, and xanthum gum. A particular example of a rate-controlling polymer for use in matrix formulations is hydroxypropylmethylcellulose of an appropriate grade (for example Methocel E4M CR (Dow) or Metolose 60 SH 4000 (Shinetzu)). Typically matrix formulations contain between about 20% w/w and about 50% w/w rate-controlling polymer, for example between about 25% w/w and about 45% w/w (based on the total weight of the tablet core).

Examples of optional binding agents include acacia, alginic acid, carboxymethylcellulose calcium, carboxymethylcellulose sodium, dextrates, dextrin, dextrose, ethylcellulose, gelatin, liquid glucose, guar gum, hydroxyethylcellulose, hydroxypropylcellulose, hydroxypropylmethylcellulose, magnesium aluminium silicate, maltodextrin, methylcellulose, polymethacrylates, polyvinylpyrrolidone, pregelatinised starch, sodium alginate, sorbitol, starch syrup, and tragacanth.

Examples of fillers include calcium carbonate, calcium phosphate, calcium sulphate, carboxymethylcellulose calcium, carboxymethylcellulose sodium, compressible sugar, confectioner's sugar, dextrates, dextrin, dextrose, dibasic calcium phosphate dihydrate, dibasic calcium phosphate, fructose, glyceryl palmitostearate, glycine, hydrogenated vegetable oil-type 1, kaolin, lactose, maize starch, magnesium carbonate, magnesium oxide, maltodextrin, mannitol, microcrystalline cellulose, polymethacrylates, potassium chloride, powdered cellulose, pregelatinised starch, sodium chloride, sorbitol, starch, sucrose, sugar spheres, talc, tribasic calcium phosphate, xylitol. A particular example of a filler for use in matrix formulations is microcrystalline cellulose. Typically matrix formulations contain between about 15% w/w and about 70% w/w of filler, for example between about 20% w/w and about 65% w/w, for example between about 30% w/w and about 60% w/w (based on the total weight of the tablet core). Formulations containing higher quantities of active ingredient, for example >140 mg of the active compound (expressed as the equivalent amount of free base), can typically contain lower quantities of filler, for example between about 10% w/w and about 60% w/w.

Examples of lubricants include calcium stearate, glyceryl monostearate, glyceryl palmitostearate, magnesium stearate, microcrystalline cellulose, sodium benzoate, sodium chloride, sodium lauryl sulphate, stearic acid, sodium stearyl fumarate, talc, and zinc stearate. A particular example of a lubricant for use in matrix formulations is magnesium stearate.

Examples of glidants include colloidal silicon dioxide, powdered cellulose, magnesium trisilicate, silicon dioxide, and talc.

Typically matrix formulations contain between about 0.5% and about 5%, for example between about 0.5% and about 2% of lubricant and/or glidant (based on the total weight of the tablet core).

Typically, where the acidic stabiliser is alginic acid (which may have a dual purpose of contributing to the sustained release profile as well as acting as a stabiliser) it may be incorporated into a sustained release matrix tablet core in the range of about 5% to about 30% w/w.

Typically the acidic stabiliser is incorporated into a sustained release matrix tablet core in the range of about 0.2% to about 20% w/w, for example about 1% to about 18%, for example about 1% to about 15%, for example about 1% to about 5%, for example about 5% to about 15% w/w, for example about 1%, for example about 3%, for example about 5%, for example about 10% w/w (based on the total weight of the tablet core).

Typically sodium bisulphate is incorporated into a sustained release matrix tablet core in the range of about 0.05% to about 2% w/w, for example about 0.2% to about 1.8%, for example about 0.5% to about 1.5% w/w, for example about 1% w/w, for example about 0.2% to about 5% (based on the total weight of the tablet core).

Typically sodium bisulphate is present in a sustained matrix tablet core in an amount of about 0.5% to about 10% w/w of the rate-controlling polymer(s), for example about 1% to about 7% w/w, for example about 1% to about 5% w/w, for example about 2% to about 4% w/w, for example about 2% w/w, or for example about 3% w/w.

In an embodiment of the present invention there is provided a sustained release pharmaceutical composition comprising a compound of formula (I) or a pharmaceutically acceptable salt or solvate thereof together with a rate-controlling polymer, a filler, and sodium bisulphate as an acidic stabiliser.

In an embodiment of the present invention there is provided a sustained release pharmaceutical composition comprising a compound of formula (I) or a pharmaceutically acceptable salt or solvate thereof together with hydroxypropylmethylcellulose as a rate-controlling polymer, microcrystalline cellulose as a filler, and sodium bisulphate as an acidic stabiliser.

Diffusion-Core Formulations

A typical presentation is a tablet, for example a coated tablet, with a hole drilled on one or both tablet surfaces. The tablet is typically round, with a diameter between 5 mm and 15 mm.

Preparation process: The drug substance is blended and wet granulated with pharmaceutically acceptable excipients, including a rate-controlling polymer. An acidic stabiliser is added directly to the other granulation excipients (e.g. alginic acid) or is first dissolved in purified water (e.g. sodium bisulphate, sulphuric acid) to produce the granulation solution. The granule is produced by conventional processing techniques, for example either a high shear or a fluid bed process, followed by drying, milling, blending and compression. The tablet core is then coated with a rate-controlling polymer, a barrier coat between the core and polymer layer may be included, as well as a final coloured aqueous film coat if required. A drying step is included after coating to remove any trapped moisture and to further enhance the stability. A hole is then drilled into either one or both of the tablet surfaces to control the rate of drug release, by controlling the surface area (e.g. a larger aperture results in a faster release rate). The aperture is generally round but can be any shape, typically in the size range of about 0.5 mm to about 8 mm, suitably about 5 mm round. The release rate can be further controlled by changes to the polymer grade and level present in the tablet core, the excipient type and level, as well as the polymer grade and level in the coat. A change in dose can be produced by increasing the level of drug substance in the granulation and decreasing the level of excipients, while adjusting the aperture size accordingly.

Suitable rate-controlling polymers for the coating may be pH-dependent or pH-independent and include:—cellulose acetate phthalate, cellulose acetate trimellitate (CAT), ethylcellulose, hydroxypropylmethylcellulose phthalate, polyvinyl acetate phthalate, polyamides, polymethacrylates, homo- and co-polymers of polylactic, polyglycolic, polyhydroxybutyric, and polyvaleric acids or esters, and polycaprolactone. Typically the rate-controlling polymer for the coating is present in an amount of between about 5% w/w and 25% w/w, for example between about 5% w/w and about 20% w/w, for example between about 5% w/w and about 15% w/w (based on the total weight of the formulation).

When necessary, the coating may be modified by addition of plasticisers or anti-tack agents. Suitable materials for this purpose include waxy materials such as glycerides, for example glyceryl monostearate, oleic acid, triethyl citrate, and DBS.

Suitable rate-controlling polymers to be included in the tablet core include:—aliphatic polyesters (homo- and co-polymers of polylactic polyglycolic, polyhydroxybutyrate, polyvaleric and polycaprolactone), alginic acid, carbomers, carboxymethyl cellulose sodium, carnauba wax, carrageenan, cellulose acetate, cellulose acetate butyrate, ethyl cellulose, glycerol monostearate, glycerol plamitostearate, glyceryl behenate, guar gum, hydroxyethylcellulose, hydroxypropylmethylcellulose, methylcellulose, polyethylene glycol, polyethylene oxide, polymethacrylates, sodium alginate, and xanthum gum. Typically diffusion-core formulations contain between about 10% w/w and about 50% w/w rate-controlling polymer in the tablet core, for example between about 10% w/w and about 45% w/w. for example between about 20% and about 35% w/w (based on the total weight of the tablet core).

Examples of suitable binding agents, fillers, lubricants and glidants are as indicated above.

Typically, where the acidic stabiliser is alginic acid (which may have a dual purpose of contributing to the sustained release profile as well as acting as a stabiliser) it may be incorporated into a diffusion-core formulation in the range of about 5% to about 30% w/w.

Typically the acidic stabiliser is incorporated into a diffusion-core formulation in the range of about 0.2% to about 20% W/W, for example about 1% to about 18%, for example about 1% to about 15%, for example about 1% to 5%, for example about 5% to about 15% w/w, for example about 1%, for example about 3%, or for example about 5%, or for example about 10% w/w (based on the total weight of the tablet core).

Typically sodium bisulphate is incorporated into a diffusion-core formulation in the range of about 0.05% to about 2% w/w, for example about 0.2% to about 1.8%, for example about 0.5% to about 1.5% w/w, for example about 1% w/w, for example about 0.2% to about 5% (based on the total weight of the tablet core).

Bead Formulations

A typical presentation is a capsule comprising of multi-layered beads. The required doses are provided by altering the fill weight in the capsules.

Preparation process: The active ingredient is dissolved in water in the presence of an acid stabiliser and binder (typically in the range of about 1% w/w to about 15% w/w, for example between about 2% to about 15%, or for example between about 1% and about 3%) and sprayed on to non-pareil beads, with a drug loading of about 20-600% w/w (for example about 30% w/w). A barrier coat is then applied onto the drug layer (for example HPMC), typically in the range of about 1-5% w/w, suitably about 2-5% w/w, for example about 3% w/w), to separate the drug layer from the rate-controlling polymer layer to further enhance the stability of the active ingredient. A rate-controlling polymer layer (for example ethylcellulose), typically between about 5% w/w and about 25% w/w, for example between about 5% w/w and about 20% w/w, for example between about 5% w/w and about 15% w/w, suitably about 8% w/w) is then applied to the beads to control the release, followed by an optional top coat (for example Opadry), to prevent sticking during further processing and for aesthetic purposes including the addition of a coloured pigment. The beads are then dried to remove any trapped moisture to further enhance the stability of the active ingredient. The beads are filled into a capsule to provide the required dose. The release profile can be further modified by changing the level of rate-controlling polymer, or mixing polymer-coated beads with immediate release beads with no polymer layer, or any other combination of coated beads.

Suitable non-pareil supports include sugar or cellulose spheres, in the range of 40-60 mesh to 14-18 mesh, preferably 35-40 mesh to 18-20 mesh sugar spheres.

Suitable binding agents include: alginic acid, carboxymethylcellulose calcium, carboxymethylcellulose sodium, dextrates, dextrin, dextrose, ethylcellulose, gelatin, liquid glucose, guar gum, hydroxyethyl cellulose, hydroxypropylcellulose, hydroxypropylmethylcellulose, magnesium aluminium silicate, maltodextrin, methylcellulose, polymethacrylates, polyvinylpyrrolidone, pregelatinised starch, sodium alginate, sorbitol, starch, syrup, and tragacanth. A particularly suitable binder is polyvinylpyrrolidone.

Suitable components for the sub-coating/barrier layer include: carboxymethylcellulose calcium, carboxymethylcellulose sodium, dextrates, dextrin, dextrose, gelatin, liquid glucose, guar gum, hydroxyethylcellulose, hydroxypropylcellulose, hydroxypropylmethylcellulose, magnesium aluminium silicate, maltodextrin, methylcellulose, polymethacrylates, polyethylene glycol, polyvinylpyrrolidone, pregelatinised starch, sodium alginate, sorbitol, sucrose starch, syrup, and tragacanth. A particularly suitable hydroxypropyl methylcellulose.

Suitable rate-controlling polymers may be pH-dependent or pH-independent and include:—cellulose acetate phthalate, cellulose acetate trimellitate (CAT), ethylcellulose, hydroxypropylmethylcellulose acetate succinate, hydroxypropylmethylcellulose phthalate, polyvinyl acetate phthalate, polyamides, polymethacrylates, and homo- and co-polymers of polylactic polyglycolic, polyhydroxybutyrate, polyvaleric and polycaprolactone.

When necessary, the erodable coating may be modified by addition of plasticisers or anti-tack agents. Suitable materials for this purpose include waxy materials such as glycerides, for example glyceryl monostearate, oleic acid, triethyl citrate, and DBS.

Typically the acidic stabiliser is incorporated into a bead formulation in the range of about 0.01% w/w to about 2% w/w, for example about 0.01% w/w to about 1.5% w/w, for example about 0.01% to about 0.5% w/w, or for example about 0.1% w/w to about 1.5% w/w (based on the total bead weight).

Typically sodium bisulphate is incorporated into a bead formulation in the range of about 0.1% W/W to about 1.5% w/w, for example about 0.1% w/w to about 1% w/w, for example about 0.2% w/w to 0.4% w/w (based on the total bead weight).

Suitable capsules include hard capsules e.g. gelatin, cellulose and low moisture capsules ranging in size from Size 0 to 4.

Alternatively, the beads can be compressed into a tablet by blending with conventional pharmaceutical excipients prior to compression. The resulting tablet can then be coated. The tablet rapidly disintegrates releasing the coated beads.

Examples of suitable fillers for such a tablet include calcium carbonate, calcium phosphate, calcium sulphate, carboxymethylcellulose calcium, carboxymethylcellulose sodium, compressible sugar, confectioner's sugar, dextrates, dextrin, dextrose, dibasic calcium phosphate dihydrate, dibasic calcium phosphate, fructose, glyceryl palmitostearate, glycine, hydrogenated vegetable oil-type 1, kaolin, lactose, maize starch, magnesium carbonate, magnesium oxide, maltodextrin, mannitol, microcrystalline cellulose, polymethacrylates, potassium chloride, powdered cellulose, pregelatinised starch, sodium chloride, sorbitol, starch, sucrose, sugar spheres, talc, tribasic calcium phosphate, and xylitol. Typically such tablets will contain between about 15% w/w and about 70% w/w of filler, for example between about 20% w/w and about 65% w/w, for example between about 30% w/w and about 60% w/w ((based on the total tablet weight).

Examples of suitable disintegrants for such a tablet (typically used in the range from about 0.5% w/w to about 10% w/w, for example about 2% w/w to about 8% w/w, for example about 3% w/w to about 7% w/w, based on the total tablet weight), include croscarmellose sodium, crospovidone, magnesium aluminium silicate, microcrystalline cellulose, methylcellulose, pregelatinised starch, and sodium starch glycollate.

Examples of suitable lubricants and glidants for such a tablet are as indicated above.

### FORMULATION EXAMPLES

The following non-limiting examples illustrate suitable sustained release pharmaceutical compositions according to the present invention.

Matrix Formulations

Typically matrix formulations will use between about 20% w/w and about 50% w/w rate-controlling polymer (based on the total weight of the tablet core), for example between about 25% w/w and about 45% w/w of rate-controlling polymer (suitably HPMC). Although typically a single rate-controlling polymer will be employed, in addition two or more (suitably two) different rate-controlling polymers may be used (including the use of the same polymer at two or more (suitably two) different grades). Although typically a single carrier will be used, in addition, two or more (suitably two) different carriers may be used. Although a combination of acidic stabilisers may be used, typically a single acidic stabiliser is used.

### Example 1

| | Amount/ |
|---|---|
| Component | unit (mg) |
| Tablet Core | |
| Formula (I)•HCl | 22.86(*) |
| Microcrystalline Cellulose (Avicel PH102) | 176.20 |
| Ph. Eur/USNF | |
| Hydroxypropylmethylcellulose (Methocel | 120.25 |
| E4M CR) Ph. Eur/USNF | |
| Sodium Bisulphate | 3.25 |
| Purified Water (removed during | qs |
| processing) Ph. Eur | |
| Magnesium Stearate Ph. Eur/USNF | 2.44 |
| TOTAL | 325.00 |
| Tablet coating | |
| Opadry White: OY-S-28876 | 13.00 |
| TOTAL | 338.00 |

(*)corresponding to 20 mg of the compound of formula (I)

### Example 2

| | Amount/ |
|---|---|
| Component | unit (mg) |
| Tablet Core | |
| Formula (I)•HCl | 45.72(*) |
| Microcrystalline Cellulose (Avicel PH102) | 159.84 |
| Ph. Eur/USNF | |
| Hydroxypropylmethylcellulose (Methocel | 113.75 |
| E4M CR) Ph. Eur/USNF | |
| Sodium Bisulphate | 3.25 |
| Purified Water (removed during | qs |
| processing) Ph. Eur | |
| Magnesium Stearate Ph. Eur/USNF | 2.44 |
| TOTAL | 325.00 |
| Tablet coating | |
| Opadry White: OY-S-28876 | 13.00 |
| TOTAL | 338.00 |

(*)corresponding to 40 mg of the compound of formula (I)

### Example 3

| | Amount/ |
|---|---|
| Component | unit (mg) |
| Tablet Core | |
| Formula (I)•HCl | 68.58(*) |
| Microcrystalline Cellulose (Avicel PH102) | 153.23 |
| Ph. Eur/USNF | |
| Hydroxypropylmethylcellulose (Methocel | 97.50 |
| E4M CR) Ph. Eur/USNF | |
| Sodium Bisulphate | 3.25 |
| Purified Water (removed during | qs |
| processing) Ph. Eur | |
| Magnesium Stearate Ph. Eur/USNF | 2.44 |
| TOTAL | 325.00 |
| Tablet coating | |
| Opadry White: OY-S-28876 | 13.00 |
| TOTAL | 338.00 |

(*)corresponding to 60 mg of the compound of formula (I)

### Example 4

| | Amount/ |
|---|---|
| Component | unit (mg) |
| Tablet Core | |
| Formula (I)•HCl | 91.44(*) |
| Microcrystalline Cellulose (Avicel PH102) | 130.37 |
| Ph. Eur/USNF | |
| Hydroxypropylmethylcellulose (Methocel | 97.50 |
| E4M CR) Ph. Eur/USNF | |
| Sodium Bisulphate | 3.25 |
| Purified Water (removed during | qs |
| processing) Ph. Eur | |
| Magnesium Stearate Ph. Eur/USNF | 2.44 |
| TOTAL | 325.00 |
| Tablet coating | |
| Opadry White: OY-S-28876 | 13.00 |
| TOTAL | 338.00 |

(*)corresponding to 80 mg of the compound of formula (I)

### Example 1B

| | Amount/ |
|---|---|
| Component | unit (mg) |
| Tablet Core | |
| Formula (I)•HCl | 22.86(*) |
| Microcrystalline Cellulose (Avicel PH102) Ph. Eur/USNF | 175.80 |
| Hydroxypropylmethylcellulose (Methocel E4M CR) Ph. Eur/ | 120.36 |
| USNF | |
| Sodium Bisulphate | 2.73 |
| Purified Water (removed during processing) Ph. Eur | qs |
| Magnesium Stearate Ph. Eur/USNF | 3.25 |
| TOTAL | 325.00 |
| Tablet coating | |
| Opadry White: YS-1R-7003 | 9.75 |
| TOTAL | 334.75 |

(*)corresponding to 20 mg of the compound of formula (I)

### Example 2B

| | Amount/ |
|---|---|
| Component | unit (mg) |
| Tablet Core | |
| Formula (I)•HCl | 45.72(*) |
| Microcrystalline Cellulose (Avicel PH102) | 175.80 |
| Ph. Eur/USNF | |
| Hydroxypropylmethylcellulose (Methocel | 120.36 |
| E4M CR) Ph. Eur/USNF | |
| Sodium Bisulphate | 2.73 |
| Purified Water (removed during | qs |
| processing) Ph. Eur | |
| Magnesium Stearate Ph. Eur/USNF | 3.25 |
| TOTAL | 325.00 |
| Tablet coating | |
| Opadry White: YS-1R-7003 | 9.75 |
| TOTAL | 334.75 |

(*)corresponding to 40 mg of the compound of formula (I)

### Example 3B

| | Amount/ |
|---|---|
| Component | unit (mg) |
| Tablet Core | |
| Formula (I)•HCl | 68.58(*) |
| Microcrystalline Cellulose (Avicel PH102) | 152.94 |
| Ph. Eur/USNF | |
| Hydroxypropylmethylcellulose (Methocel | 97.50 |
| E4M CR) Ph. Eur/USNF | |
| Sodium Bisulphate | 2.73 |
| Purified Water (removed during | qs |
| processing) Ph. Eur | |
| Magnesium Stearate Ph. Eur/USNF | 3.25 |
| TOTAL | 325.00 |
| Tablet coating | |
| Opadry White: YS-1R-7003 | 9.75 |
| TOTAL | 334.75 |

(*)corresponding to 60 mg of the compound of formula (I)

### Example 4B

| | Amount/ |
|---|---|
| Component | unit (mg) |
| Tablet Core | |
| Formula (I)•HCl | 91.44(*) |
| Microcrystalline Cellulose (Avicel PH102) | 130.08 |
| Ph. Eur/USNF | |
| Hydroxypropylmethylcellulose (Methocel E4M | 97.50 |
| CR) Ph. Eur/USNF | |
| Sodium Bisulphate | 2.73 |
| Purified Water (removed during | qs |
| processing) Ph. Eur | |
| Magnesium Stearate Ph. Eur/USNF | 3.25 |
| TOTAL | 325.00 |
| Tablet coating | |
| Opadry White: YS-1R-7003 | 9.75 |
| TOTAL | 334.75 |

(*)corresponding to 80 mg of the compound of formula (I)

### Example 1C

| | Amount/ |
|---|---|
| Component | unit (mg) |
| Tablet Core | |
| Formula (I)•HCl | 22.86(*) |
| Microcrystalline Cellulose (Avicel PH102) | 175.39 |
| Ph. Eur/USNF | |
| Hydroxypropylmethylcellulose (Methocel | 120.25 |
| E4M CR) Ph. Eur/USNF | |
| Sodium Bisulphate | 3.25 |
| Purified Water (removed during | qs |
| processing) Ph. Eur | |
| Magnesium Stearate Ph. Eur/USNF | 3.25 |
| TOTAL | 325.00 |
| Tablet coating | |
| Opadry White: YS-1R-7003 | 13.00 |
| TOTAL | 338.00 |

(*)corresponding to 20 mg of the compound of formula (I)

### Example 2C

| | Amount/ |
|---|---|
| Component | unit (mg) |
| Tablet Core | |
| Formula (I)•HCl | 45.72(*) |
| Microcrystalline Cellulose (Avicel PH102) | 159.03 |
| Ph. Eur/USNF | |
| Hydroxypropylmethylcellulose (Methocel | 113.75 |
| E4M CR) Ph. Eur/USNF | |
| Sodium Bisulphate | 3.25 |
| Purified Water (removed during | qs |
| processing) Ph. Eur | |
| Magnesium Stearate Ph. Eur/USNF | 3.25 |
| TOTAL | 325.00 |
| Tablet coating | |
| Opadry White: YS-1R-7003 | 13.00 |
| TOTAL | 338.00 |

(*)corresponding to 40 mg of the compound of formula (I)

### Example 3C

| | Amount/ |
|---|---|
| Component | unit (mg) |
| Tablet Core | |
| Formula (I)•HCl | 68.58(*) |
| Microcrystalline Cellulose (Avicel PH102) | 152.42 |
| Ph. Eur/USNF | |
| Hydroxypropylmethylcellulose (Methocel | 97.50 |
| E4M CR) Ph. Eur/USNF | |
| Sodium Bisulphate | 3.25 |
| Purified Water (removed during | qs |
| processing) Ph. Eur | |
| Magnesium Stearate Ph. Eur/USNF | 3.25 |
| TOTAL | 325.00 |
| Tablet coating | |
| Opadry White: YS-1R-7003 | 13.00 |
| TOTAL | 338.00 |

(*)corresponding to 60 mg of the compound of formula (I)

### Example 4C

| | Amount/ |
|---|---|
| Component | unit (mg) |
| Tablet Core | |
| Formula (I)•HCl | 91.44(*) |
| Microcrystalline Cellulose (Avicel PH102) | 129.56 |
| Ph. Eur/USNF | |
| Hydroxypropylmethylcellulose (Methocel | 97.50 |
| E4M CR) Ph. Eur/USNF | |
| Sodium Bisulphate | 3.25 |
| Purified Water (removed during | qs |
| processing) Ph. Eur | |
| Magnesium Stearate Ph. Eur/USNF | 3.25 |
| TOTAL | 325.00 |
| Tablet coating | |
| Opadry White: YS-1R-7003 | 13.00 |
| TOTAL | 338.00 |

(*)corresponding to 80 mg of the compound of formula (I)

### Example 5

| | Amount/ |
|---|---|
| Component | unit (mg) |
| Tablet Core | |
| Formula (I)•HCl | 11.43(*) |
| Microcrystalline Cellulose (Avicel PH102) | 160.87 |
| Ph. Eur/USNF | |
| Hydroxypropylmethylcellulose (Methocel | 146.25 |
| E4M CR) Ph. Eur/USNF | |
| Sodium Bisulphate | 3.25 |
| Purified Water (removed during | qs |
| processing) Ph. Eur | |
| Magnesium Stearate Ph. Eur/USNF | 3.25 |
| TOTAL | 325.05 |
| Tablet coating | |
| Opadry White: YS-1R-7003 | 9.75 |
| TOTAL | 334.80 |

(*)corresponding to 10 mg of the compound of formula (I)

### Example 6

| | Amount/ |
|---|---|
| Component | unit (mg) |
| Tablet Core | |
| Formula (I)•HCl | 11.43(*) |
| Microcrystalline Cellulose (Avicel PH102) | 169.10 |
| Ph. Eur/USNF | |
| Hydroxypropylmethylcellulose (Methocel | 137.97 |
| E4M CR) Ph. Eur/USNF | |
| Sodium Bisulphate | 3.25 |
| Purified Water (removed during | qs |
| processing) Ph. Eur | |
| Magnesium Stearate Ph. Eur/USNF | 3.25 |
| TOTAL | 325.00 |
| Tablet coating | |
| Opadry White: YS-1R-7003 | 13.00 |
| TOTAL | 338.00 |

(*)corresponding to 10 mg of the compound of formula (I)

Examples 1 to 6 above were prepared by a process similar to the following general process: The drug substance is blended and wet granulated with the pharmaceutically acceptable excipients described, including HPMC as the rate-controlling polymer. The acidic stabiliser (sodium bisulphate) is first dissolved in purified water to produce the granulation solution, and the granule is then produced by conventional processing techniques, for example either high shear or a fluid bed process, followed by drying, milling, blending, compression into a tablet, and finally aqueous film-coating.

Examples 7 and 8 below (prepared by an analogous method to Examples 1 to 5 above) are further examples of oral formulations; these were uncoated tablets, containing 20 mg and 40 mg of the compound of formula (I) as the hydrochloride salt. The tablets were manufactured using a wet granulation process in a fluid bed granulator. The release rate-controlling function is provided by Hypromellose, which is blended with the active ingredient (compound of formula (I) as its hydrochloride salt) and the microcrystalline cellulose filler, then granulated in a fluid bed granulator by spraying an aqueous sulphuric acid solution.

| | | Amount/ |
|---|---|---|
| Example | Components | unit (mg) |
| 7 | Formula (I)•HCl | 45.72(*) |
| | Microcrystalline Cellulose (Avicel | 70.30 |
| | PH-102) EP/USP | |
| | Hydroxypropyl Methylcellulose (Methocel | 81.26 |
| | E4M CR) EP/USP | |
| | Sulphuric Acid EP/USP | 2.72 |
| | Purified Water (removed during | qs |
| | processing) EP/USP | |
| | Lactose monohydrate spray dried EP/USP | 121.75 |
| | Magnesium stearate EP/USP | 3.25 |
| | TOTAL | 325.00 |
| 8 | Formula (I)•HCl | 22.86(^{§}) |
| | Microcrystalline Cellulose (Avicel | 93.16 |
| | PH-102) EP/USP | |
| | Hydroxypropyl Methylcellulose (Methocel | 81.26 |
| | E4M CR) EP/USP | |
| | Sulphuric Acid EP/USP | 2.72 |
| | Purified Water (removed during | qs |
| | processing) EP/USP | |
| | Lactose monohydrate spray dried EP/USP | 121.75 |
| | Magnesium stearate EP/USP | 3.25 |
| | TOTAL | 325.00 |

(*)corresponding to 40.00 mg of the compound of formula (I)
(^{§})corresponding to 20.00 mg of the compound of formula (I)

Examples 9 to 16 below were prepared by an analogous method to Examples 1 to 6.

### Example 9

| | Amount/ |
|---|---|
| Components | unit (mg) |
| Formula (I)•HCl | 22.75 |
| Microcrystalline Cellulose (Avicel PH102) | 165.75 |
| Hydroxypropylmethylcellulose (Methocel E4M CR) | 130.00 |
| Sodium Bisulphate | 3.25 |
| Purified Water (removed during processing) | qs |
| Magnesium Stearate | 3.25 |
| Opadry White: YS-1R-7003 | 9.75 |
| TOTAL | 334.75 |

### Example 10

| | Amount/ |
|---|---|
| Components | unit (mg) |
| Formula (I)•HCl | 45.73 |
| Microcrystalline Cellulose (Avicel PH102) | 177.52 |
| Hydroxypropylmethylcellulose (Methocel E4M CR) | 97.50 |
| Sulphuric Acid | 1.00 |
| Purified Water (removed during processing) | qs |
| Magnesium Stearate | 3.25 |
| TOTAL | 325.00 |

### Example 11

| | Amount/ |
|---|---|
| Components | unit (mg) |
| Formula (I)•HCl | 45.73 |
| Microcrystalline Cellulose (Avicel PH102) | 193.77 |
| Hydroxypropylmethylcellulose (Methocel E4M CR) | 81.25 |
| Sulphuric Acid | 1.00 |
| Purified Water (removed during processing) | qs |
| Magnesium Stearate | 3.25 |
| TOTAL | 325.00 |

### Example 12

| | Amount/ |
|---|---|
| Components | unit (mg) |
| Formula (I)•HCl | 91.33 |
| Microcrystalline Cellulose (Avicel PH102) | 129.67 |
| Hydroxypropylmethylcellulose (Methocel E4M CR) | 97.50 |
| Sodium Bisulphate | 3.25 |
| Purified Water (removed during processing) | qs |
| Magnesium Stearate | 3.25 |
| Opadry White: YS-1R-7003 | 9.75 |
| TOTAL | 334.75 |

### Example 13

| | Amount/ |
|---|---|
| Components | unit (mg) |
| Formula (I)•HCl | 137.15 |
| Microscrystalline Cellulose (Avicel PH102) | 84.37 |
| Hydroxypropylmethylcellulose (Methocel E4M CR) | 97.50 |
| Sodium Bisulphate | 2.73 |
| Purified Water (removed during processing) | qs |
| Magnesium Stearate | 3.25 |
| Opadry White: YS-1R-7003 | 9.75 |
| TOTAL | 334.75 |

### Example 14

| | Amount/ |
|---|---|
| Components | unit (mg) |
| Formula (I)•HCl | 22.75 |
| Microcrystalline Cellulose (Avicel PH102) | 176.07 |
| Hydroxypropylmethylcellulose (Methocel E4M CR) | 97.50 |
| Hydroxypropylmethylcellulose (Methocel E5 LV) | 22.76 |
| Sodium Bisulphate | 2.67 |
| Purified Water (removed during processing) | qs |
| Magnesium Stearate | 3.25 |
| Opadry White: YS-1R-7003 | 9.75 |
| TOTAL | 334.75 |

### Example 15

| | Amount/ |
|---|---|
| Components | unit (mg) |
| Formula (I)•HCl | 11.44 |
| Microscrystalline Cellulose (Avicel PH102) | 99.12 |
| Lactose Monohydrate | 110.44 |
| Hydroxypropylmethylcellulose (Methocel E4M CR) | 97.50 |
| Sodium Bisulphate | 3.25 |
| Purified Water (removed during processing) | qs |
| Magnesium Stearate | 3.25 |
| Opadry White: YS-1R-7003 | 9.75 |
| TOTAL | 334.75 |

### Example 16

| | Amount/ |
|---|---|
| Components | unit (mg) |
| Formula (I)•HCl | 91.46 |
| Microcrystalline Cellulose (Avicel PH102) | 99.12 |
| Lactose Monohydrate | 30.42 |
| Hydroxypropylmethylcellulose (Methocel E4M CR) | 97.50 |
| Sodium Bisulphate | 3.25 |
| Purified Water (removed during processing) | qs |
| Magnesium Stearate | 3.25 |
| Opadry White: YS-1R-7003 | 9.75 |
| TOTAL | 334.75 |

Examples 17 to 19 were prepared by an analogous method to Examples 1 to 6.

### Example 17

| | Amount/ |
|---|---|
| Components | unit (mg) |
| Formula (I)•HCl | (*)114.30 |
| Microcrystalline Cellulose (Avicel PH102) | 107.51 |
| Hydroxypropylmethylcellulose 2910 | 97.50 |
| Sodium Bisulphate | 3.25 |
| Purified Water (removed during processing) | qs |
| Magnesium Stearate | 2.44 |
| TOTAL | 325.00 |
| Opadry White: OY-S-28876 | 13.00 |
| TOTAL | 338.00 |

(*)corresponding to 100 mg of the compound of formula (I)

### Example 18

| | Amount/ |
|---|---|
| Components | unit (mg) |
| Formula (I)•HCl | (*)137.16 |
| Microcrystalline Cellulose (Avicel PH102) | 84.65 |
| Hydroxypropylmethylcellulose 2910 | 97.50 |
| Sodium Bisulphate | 3.25 |
| Purified Water (removed during processing) | qs |
| Magnesium Stearate | 2.44 |
| TOTAL | 325.00 |
| Opadry White: OY-S-28876 | 13.00 |
| TOTAL | 338.00 |

(*)corresponding to 120 mg of the compound of formula (I).

### Example 19

| | Amount/ |
|---|---|
| Components | unit (mg) |
| Formula (I)•HCl | (*)160.02 |
| Microcrystalline Cellulose (Avicel PH102) | 61.79 |
| Hydroxypropylmethylcellulose 2910 | 97.50 |
| Sodium Bisulphate | 3.25 |
| Purified Water (removed during processing) | qs |
| Magnesium Stearate | 2.44 |
| TOTAL | 325.00 |
| Opadry White: OY-S-28876 | 13.00 |
| TOTAL | 338.00 |

(*)corresponding to 140 mg of the compound of formula (I)

Alternative formulations to those of Examples 17 to 19 containing 100 mg, 120 mg and 140 mg respectively of the compound of formula (I) (using the free base or a pharmaceutically acceptable salt or solvate thereof, especially the hydrochloride salt) may be prepared by an analogous process but with other suitable changes, for example using an increased tablet weight, including the addition of a binder (for example polyvinylpyrrolidine or an alternative binder), using a greater proportion of lubricant, together with any other suitable changes to obtain an acceptable sustained release tablet.

Examples 20 to 23 were prepared by an analogous method to Examples 1 to 6.

### Example 20

| | Amount/ |
|---|---|
| Components | unit (mg) |
| Formula (I)•HCl | (*)114.30 |
| Microcrystalline Cellulose (Avicel PH102) | 106.70 |
| Hydroxypropylmethylcellulose 2910 | 97.50 |
| Sodium Bisulphate | 3.25 |
| Purified Water (removed during processing) | qs |
| Magnesium Stearate | 3.25 |
| TOTAL | 325.00 |
| Opadry White: OY-S-28876 | 13.00 |
| TOTAL | 338.00 |

(*)corresponding to 100 mg of the compound of formula (I)

### Example 21

| | Amount/ |
|---|---|
| Components | unit (mg) |
| Formula (I)•HCl | (*)137.16 |
| Microcrystalline Cellulose (Avicel PH102) | 83.84 |
| Hydroxypropylmethylcellulose 2910 | 97.50 |
| Sodium Bisulphate | 3.25 |
| Purified Water (removed during processing) | qs |
| Magnesium Stearate | 3.25 |
| TOTAL | 325.00 |
| Opadry White: OY-S-28876 | 13.00 |
| TOTAL | 338.00 |

(*)corresponding to 120 mg of the compound of formula (I).

### Example 22

| | Amount/ |
|---|---|
| Components | unit (mg) |
| Formula (I)•HCl | (*)160.02 |
| Microcrystalline Cellulose (Avicel PH102) | 60.98 |
| Hydroxypropylmethylcellulose 2910 | 97.50 |
| Sodium Bisulphate | 3.25 |
| Purified Water (removed during processing) | qs |
| Magnesium Stearate | 3.25 |
| TOTAL | 325.00 |
| Opadry White: OY-S-28876 | 13.00 |
| TOTAL | 338.00 |

(*)corresponding to 140 mg of the compound of formula (I)

### Example 23

| | Amount/ |
|---|---|
| Components | unit (mg) |
| Formula (I)•HCl | (*)182.88 |
| Microcrystalline Cellulose (Avicel PH102) | 38 [symbol] |
| Hydroxypropylmethylcellulose 2910 | 97 [symbol] |
| Sodium Bisulphate | 3.25 |
| Purified Water (removed during processing) | qs |
| Magnesium Stearate | 3.25 |
| TOTAL | 325.00 |
| Opadry White: OY-S-28876 | 13.00 |
| TOTAL | 338.00 |

(*)corresponding to 160 mg of the compound of formula (I)

Examples 24 to 27 containing higher amounts of the active ingredient were prepared by an analogous method to Examples 1 to 6.

### Example 24

| | Amount/ |
|---|---|
| Components | unit (mg) |
| Formula (I)•HCl | (*)205.74 |
| Microcrystalline Cellulose (Avicel PH102) | 42.46 |
| Hydroxypropylmethylcellulose 2910 | 109.50 |
| Sodium Bisulphate | 3.65 |
| Purified Water (removed during processing) | qs |
| Magnesium Stearate | 3.65 |
| TOTAL | 365.00 |
| Opadry White: OY-S-28876 | 15.00 |
| TOTAL | 380.00 |

(*)corresponding to 180 mg of the compound of formula (I)

### Example 25

| | Amount/ |
|---|---|
| Components | unit (mg) |
| Formula (I)•HCl | (*)228.60 |
| Microcrystalline Cellulose (Avicel PH102) | 46.80 |
| Hydroxypropylmethylcellulose 2910 | 121.50 |
| Sodium Bisulphate | 4.05 |
| Purified Water (removed during processing) | qs |
| Magnesium Stearate | 4.05 |
| TOTAL | 405.00 |
| Opadry White: OY-S-28876 | 16.00 |
| TOTAL | 421.00 |

(*)corresponding to 200 mg of the compound of formula (I)

### Example 26

| | Amount/ |
|---|---|
| Components | unit (mg) |
| Formula (I)•HCl | (*)251.46 |
| Microcrystalline Cellulose (Avicel PH102) | 49.10 |
| Hydroxypropylmethylcellulose 2910 | 132.60 |
| Sodium Bisulphate | 4.42 |
| Purified Water (removed during processing) | qs |
| Magnesium Stearate | 4.42 |
| TOTAL | 442.00 |
| Opadry White: OY-S-28876 | 18.00 |
| TOTAL | 460.00 |

(*)corresponding to 220 mg of the compound of formula (I)

### Example 27

| | Amount/ |
|---|---|
| Components | unit (mg) |
| Formula (I)•HCl | (*)274.32 |
| Microcrystalline Cellulose (Avicel PH102) | 57.17 |
| Hydroxypropylmethylcellulose 2910 | 146.25 |
| Sodium Bisulphate | 4.88 |
| Purified Water (removed during processing) | qs |
| Magnesium Stearate | 4.88 |
| TOTAL | 487.50 |
| Opadry White: OY-S-28876 | 19.50 |
| TOTAL | 507.00 |

(*)corresponding to 240 mg of the compound of formula (I)

Examples 28 and 29 were prepared by an analogous method to Examples 1 to 6.

| Components | Example 28 | Example 29 |
|---|---|---|
| Tablet Core | | |
| Formula (I)•HCl | 91.44¹ | 91.44¹ |
| Microcrystalline Cellulose | 178.31 | 32.06 |
| Hydroxypropyl Methylcellulose 2910 | 48.75 | 195.00 |
| Magnesium Stearate | 3.25 | 3.25 |
| Sodium Hydrogen Sulphate | 3.25 | 3.25 |
| Purified Water² | — | — |
| Coat | | |
| Opadry ® White YS-1R-7003 | 13.00 | 13.00 |
| Purified water² | — | — |
| Total unit dose | 338.00 | 338.00 |

¹Corresponding to 80 mg of the compound of formula (I).
²Removed during processing.

The rate of in vitro drug release from the sustained release pharmaceutical compositions according to the present invention is determined by a drug release test according to USP <724> Extended Release Articles, using the USP rotating paddle apparatus (Apparatus 2) and the Acceptance Criteria Table 1.

Table 2 shows the dissolution data (% of compound of formula (I) released) for certain of the above-mentioned matrix formulations using the USP II method, a paddle speed of 50 rpm, and a phosphate buffer (pH 6.8, 0.05M).

TABLE 2

| Time (hours) | Example 10 | Example 11 | Example 11B * |
|---|---|---|---|
| 0 | 0 | 0 | 0 |
| 1 | 22 | 24 | 29 |
| 2 | 32 | 35 | 42 |
| 4 | 46 | 51 | 60 |
| 8 | 65 | 69 | 81 |

(* this formulation has the same components as Example 11, but is compressed into a capsule shaped tablet).

Diffusion-Core Formulations

The formulations are prepared by a general process as indicated above. For Examples 30 to 34 the tablet core consists of a platform granule, which is blended with extragranular excipients before compression; each example contains the equivalent of 40 mg of the compound of formula (I) as the active ingredient. HPMC is used as the polymer in the core. An aqueous film-coat (Opadry) is applied to the tablet core and acts as a barrier between the core and outer layer. Examples 30 to 33 have a drilled 5 mm hole on both the upper and lower tablet surfaces.

### Example 30

| | Amount/ |
|---|---|
| Components | unit (mg) |
| Formula (I)•HCl | 45.72 (*) |
| Lactose Monohydrate (Pharmatose DCL) | 210.03 |
| Hydroxypropylmethylcellulose (Methocel K100 LV CR) | 65.00 |
| Concentrated Sulphuric Acid | 1.00 |
| Purified Water (removed during processing) | qs |
| Magnesium Stearate | 3.25 |
| Core weight | 325.00 |
| Surelease E-7-19010 | 39.00 |
| Opadry White YS-1-7003 | 10.9 |
| TOTAL | 374.9 |

### Example 31

| | Amount/ |
|---|---|
| Components | unit (mg) |
| Formula (I)•HCl | 45.72 |
| Lactose Monohydrate (Pharmatose DCL) | 178.53 |
| Hydroxypropylmethylcellulose (Methocel K100 LV CR) | 65.00 |
| Alginic Acid | 32.50 |
| Purified Water (removed during processing) | qs |
| Magnesium Stearate | 3.25 |
| Core weight | 325.00 |
| Surelease E-7-19010 | 39.00 |
| Opadry White YS-1-7003 | 10.9 |
| TOTAL | 374.9 |

### Example 32

| | Amount/ |
|---|---|
| Components | unit (mg) |
| Formula (I)•HCl | 45.72 |
| Lactose Monohydrate (Pharmatose DCL) | 211.03 |
| Hydroxypropylmethylcellulose (Methocel K100 LV CR) | 32.50 |
| Alginic Acid | 32.50 |
| Purified Water (removed during processing) | qs |
| Magnesium Stearate | 3.25 |
| Total Core Weight | 325.00 |
| Opadry White YS-1-7003 | 9.75 |
| Surelease E-7-19010 | 66.95 |
| TOTAL | 401.70 |

### Example 33

| | Amount/ |
|---|---|
| Components | unit (mg) |
| Formula (I)•HCl | 45.72 |
| Lactose Monohydrate (Pharmatose DCL) | 178.53 |
| Hydroxypropylmethylcellulose (Methocel K100 LV CR) | 65.00 |
| Alginic Acid | 32.50 |
| Purified Water (removed during processing) | qs |
| Magnesium Stearate | 3.25 |
| Total Core Weight | 325.00 |
| Opadry White YS-1-7003 | 9.75 |
| Surelease E-7-19010 | 66.95 |
| TOTAL | 401.70 |

Example 34 is identical to Example 32 in terms of components and quantities, but differs in hole size (drilled hole 4 mm on both upper and lower tablet surface).

Table 3 shows the dissolution data (% of compound of formula (I) released) for certain of the above-mentioned diffusion-core Examples (USP II apparatus, 50 rpm, phosphate buffer pH 6.8).

TABLE 3

| Time (hours) | Example 32 | Example 34 | Example 33 |
|---|---|---|---|
| 0 | 0 | 0 | 0 |
| 1 | 11 | 7 | 8 |
| 2 | 20 | 12 | 15 |
| 4 | 39 | 24 | 28 |
| 8 | 79 | 50 | 54 |

Bead Formulations

The formulations are prepared by a general process as indicated above. For each of Examples 35 to 39 the formulation contains the equivalent of 40 mg of the compound of formula (I) as the active ingredient, whereas for Example 40 the formulation contains the equivalent of 80 mg of the compound of formula (I) as the active ingredient.

### Example 35

| | Amount/ |
|---|---|
| Components | unit (mg) |
| Formula (I)•HCl | 45.72 |
| Sugar spheres 35-40 mesh | 79.97 |
| Surelease (E-7-19010) | 8.71 |
| Povidone | 4.06 |
| Hydroxypropyl Methylcellulose (Methocel E5 LV) | 3.34 |
| Opadry White (OY-S-7335) | 2.90 |
| Sodium Bisulphate monohydrate | 0.44 |
| Purified Water (removed during processing) | qs |
| Hard gelatine capsule size 0 | 95.00 |
| TOTAL | 240.14 |

### Example 36

| | Amount/ |
|---|---|
| Components | unit (mg) |
| Formula (I)•HCl | 45.72 |
| Sugar spheres 20-2T | 143.21 |
| Surelease (E-7-19010) | 13.00 |
| Povidone | 6.00 |
| Sucrose | 5.00 |
| Opadry White (OY-S-7335) | 4.50 |
| Sulphuric Acid | 0.07 |
| Purified Water (removed during processing) | qs |
| Hard gelatine capsule size 0 | 95.00 |
| TOTAL | 312.50 |

### Example 37

| | Amount/ |
|---|---|
| Components | unit (mg) |
| Formula (I)•HCl | 45.72 |
| Sugar spheres 20-2T | 143.21 |
| Surelease (E-7-19010) | 17.50 |
| Povidone | 6.00 |
| Sucrose | 5.00 |
| Opadry White (OY-S-7335) | 4.50 |
| Sulphuric Acid | 0.07 |
| Purified Water (removed during processing) | — |
| Hard gelatine capsule size 0 | 95.00 |
| TOTAL | 317.00 |

### Example 38

| | Amount/ |
|---|---|
| Components | unit (mg) |
| Formula (I)•HCl | 45.72 |
| Sugar spheres 20-2T | 143.21 |
| Surelease (E-7-19010) | 22.50 |
| Povidone | 6.00 |
| Sucrose | 5.00 |
| Opadry White (OY-S-7335) | 4.50 |
| Sulphuric Acid | 0.07 |
| Purified Water (removed during processing) | qs |
| Hard gelatine capsule size 0 | 95.00 |
| TOTAL | 322.00 |

### Example 39

| | Amount/ |
|---|---|
| Components | unit (mg) |
| Formula (I)•HCl | 45.72 |
| Sugar spheres 20-2T | 143.21 |
| Surelease (E-7-19010) | 38.20 |
| Povidone | 6.00 |
| Sucrose | 5.00 |
| Opadry White (OY-S-7335) | 4.80 |
| Sulphuric Acid | 0.07 |
| Purified Water (removed during processing) | qs |
| Hard gelatine capsule size 0 | 95.00 |
| TOTAL | 338.00 |

### Example 40

| | Amount/ |
|---|---|
| Components | unit (mg) |
| Formula (I)•HCl | 91.44 |
| Sugar spheres 35-40 mesh | 157.13 |
| Surelease (E-7-19010) | 23.67 |
| Povidone | 7.99 |
| Hydroxypropyl Methylcellulose (Methocel E5 LV) | 8.88 |
| Opadry White (OY-S-7335) | 5.92 |
| Sodium Bisulphate monohydrate | 0.89 |
| Purified Water (removed during processing) | qs |
| Hard gelatine capsule size 0 | 95.00 |
| TOTAL | 390.92 |

### Example 41

Using mixed beads in the ratio 90:10

| | Amount/ |
|---|---|
| Components | unit (mg) |
| Uncoated | |
| Formula (I)•HCl | 4.57 |
| Sugar spheres 20-2T | 14.32 |
| Povidone | 0.60 |
| Sucrose | 0.50 |
| Sulphuric Acid | 0.01 |
| Purified Water (removed during processing) | qs |
| Coated | |
| Formula (I)•HCl | 41.15 |
| Sugar spheres 20-2T | 128.88 |
| Surelease (E-7-19010) | 11.71 |
| Povidone | 5.40 |
| Sucrose | 4.50 |
| Opadry White (OY-S-7335) | 4.05 |
| Sulphuric Acid | 0.06 |
| Purified Water (removed during processing) | qs |
| Hard gelatine capsule size 0 | 95.00 |
| TOTAL | 310.75 |

Example 42 illustrates another possible formulation (a disintegrating tablet containing beads with an HPMC-containing subcoat as in Example 35) which may be made by a general process as described above.

### Example 42

| | Amount/ |
|---|---|
| Components | unit (mg) |
| Formula (I)•HCl | 45.72 |
| Sugar spheres 35-40 mesh | 79.97 |
| Surelease (E-7-19010) | 8.71 |
| Povidone | 4.06 |
| Hydroxypropyl Methylcellulose (Methocel E5 LV) | 3.34 |
| Opadry White (OY-S-7335) | 2.90 |
| Sodium Bisulphate monohydrate | 0.44 |
| Purified Water (removed during processing) | qs |
| Microscrystalline Cellulose (Avicel PH102) | 286.86 |
| Sodium Starch Glycolate | 13.50 |
| Magnesium stearate | 4.50 |
| Opadry White (OY-S-7335) | 13.50 |
| TOTAL | 463.50 |

Table 4 illustrates dissolution data (% compound of formula (I) dissolved) for the bead formulation Examples using USP II apparatus (50 rpm basket speed; phosphate buffer pH 6.8).

TABLE 4

| | Exam- | Exam- | Exam- | Exam- | Exam- | Exam- |
|---|---|---|---|---|---|---|
| Time | ple | ple | ple | ple | ple | ple |
| (hours) | 35 | 36 | 37 | 38 | 39 | 41 |
| 0 | 0 | 0 | 0 | 0 | 0 | 0 |
| 1 | 10 | 8 | 7 | 1 | 0 | 17 |
| 2 | 26 | 20 | 15 | 5 | 2 | 28 |
| 4 | 51 | 40 | 33 | 16 | 7 | 47 |
| 8 | 78 | 61 | 55 | 32 | 18 | 66 |

In a further aspect of the present invention there is provided a sustained release pharmaceutical composition for oral administration comprising a compound of formula (I) or a pharmaceutically acceptable derivative thereof as active ingredient wherein in a phosphate buffer (pH 6.8) in USPII apparatus at the discriminating paddle speed of about 50 rpm, between about 5% and about 35%, for example between about 15% and about 35% of the compound of formula (I) is dissolved within 1 hour, between about 15% to about 65%, for example between about 20% to about 65%, for example between about 35% to about 65% of the compound of formula (I) is dissolved within 4 hours, and not less than about 30%, for example not less than about 50%, for example not less than about 55% of the compound of formula (I) is dissolved within 8 hours.

In a further aspect of the present invention there is provided a sustained release pharmaceutical composition for oral administration comprising a compound of formula (I) or a pharmaceutically acceptable derivative thereof as active ingredient wherein in a phosphate buffer (pH 6.8) in USPII apparatus at the discriminating paddle speed of about 50 rpm, between about 25% and about 45% of the compound of formula (I) is dissolved within 1 hour, between about 60% to about 85% of the compound of formula (I) is dissolved within 4 hours, and not less than about 80% of the compound of formula (I) is dissolved within 8 hours.

In Vivo Pharmacokinetic Characteristics

Sustained release compositions according to the present invention are intended to provide effective release of the active ingredient (the compound of formula (I)) over a sufficient time period to allow for once-daily dosing in the treatment of the relevant medical condition or disorder.

The particular pharmacokinetic characteristics referred to hereinafter will be calculated as either mean values or median values, as appropriate (but preferably as indicated below) for each characteristic, from the individual values obtained for each person (whether a human volunteer or a human patient) studied. In general, such mean or median values will be calculated using trials containing at least 8 people, preferably at least 15 people, and typically between about 10 and about 35 people, more typically between about 15 and about 35 people.

The compound of formula (I) is extremely soluble in an acidic environment (approx. 150 mg/ml) and therefore a good rate-controlling delivery system is required to modify the Cmax achieved using an immediate-release formulation.

As compared to a corresponding immediate-release formulation, the compositions of the present invention have been shown to have a reduced Cmax (maximum observed plasma concentration) and a prolonged Tmax (time to maximum observed plasma concentration) but a comparable AUCinfinity (area under concentration-time curve for the time period 0-infinity).

Following oral administration of a single unit dose sustained release composition of the present invention comprising the compound of formula (I) in the dosage range of 20-160 mg, or at a dose of 10 mg, (typically using the hydrochloride salt of the compound of formula (I)), maximum plasma concentrations of the compound of formula (I) for each dose strength are typically expected to be observed (Tmax, median) between about 3 and about 12 hours, for example between about 3 and about 10 hours, for example between about 4 hours and about 8 hours, for example between about 5 hours and about 7 hours after dosing. Thereafter plasma concentrations are expected to decrease in an apparent monoexponential manner.

Thus, in an embodiment of the present invention, there is provided a sustained release pharmaceutical composition for oral administration comprising a compound of formula (I) or a pharmaceutically acceptable salt or solvate thereof as active ingredient, wherein single dose administration in human subjects of a unit dose of the compound of formula (I) between 20 mg and 160 mg provides a Tmax between about 3 and about 12 hours, for example between about 3 and about 10 hours, for example between about 4 hours and about 8 hours, for example between about 5 hours and about 7 hours.

The terminal phase half life (T½, median) is typically expected to be between about 10 and about 18 hours over this same dosage range.

Thus, in an embodiment of the present invention, there is provided a sustained release pharmaceutical composition for oral administration comprising a compound of formula (I) or a pharmaceutically acceptable salt or solvate thereof as active ingredient, wherein single dose administration in human subjects of a unit dose of the compound of formula (I) between 20 mg and 160 mg provides a T½ between about 10 hours and about 18 hours.

Typical mean Cmax values following administration of a single unit dose of a sustained release composition according to the present invention (dosage of the compound of formula (I) as the free base) are expected to be within the ranges as set out in Table 5 below.

TABLE 5

| | Mean Cmax in range |
|---|---|
| Dosage (mg) | (ng/mL) |
| 20 | 50-120 |
| 40 | 80-180 |
| 60 | 120-300 |
| 80 | 180-350 |
| 100 | 240-400 |
| 120 | 320-530 |
| 140 | 400-640 |
| 160 | 480-750 |

Typical mean Cmax values following repeated once-daily administration over a period of time to achieve steady-state (typically over 7, 14 or 28 days) of a unit dose of a sustained release composition according to the present invention (dosage of the compound of formula (I) as the free base) are expected to be within the ranges as set out in Table 6 below.

TABLE 6

| | Mean Cmax in range |
|---|---|
| Dosage (mg) | (ng/mL) |
| 20 | 75-180 |
| 40 | 120-270 |
| 60 | 180-450 |
| 80 | 270-525 |
| 100 | 320-600 |
| 120 | 480-795 |
| 140 | 600-960 |
| 160 | 720-1120 |

Thus, in further embodiments of the present invention, there is provided a sustained release pharmaceutical composition for oral administration comprising a compound of formula (I) or a pharmaceutically acceptable salt or solvate thereof as active ingredient, wherein single dose administration in human subjects of each dosage set out in Table 5 provides a mean Cmax value within the corresponding range for that dosage as set out in Table 5.

In further embodiments of the present invention, there is provided a sustained release pharmaceutical composition for oral administration comprising a compound of formula (I) or a pharmaceutically acceptable salt or solvate thereof as active ingredient, wherein repeat once-daily dose administration in human subjects of each dosage set out in Table 6 provides a mean Cmax value within the corresponding range for that dosage as set out in Table 6.

Various of the formulations of the above-mentioned Examples have been administered as a single unit dose to human subjects (volunteers or patients). Tables 7 and 8 below set out the pharmacokinetic data observed.

TABLE 7

| | Number of | Median | Mean¹ Cmax | Median |
|---|---|---|---|---|
| Example | patients (n) | Tmax (h) | (ng/ml) | T½ (h) |
| 1B | 27 | 7 | 62 | 13.7 |
| 2B | 40 | 7 | 122 | 14.4 |
| 3B | 27 | 5 | 175 | 13.4 |
| 4B | 26 | 6 | 237 | 14.7 |

¹Geometric mean

TABLE 8

| | Number of | Median Cmax | Median |
|---|---|---|---|
| Example | patients (n) | (ng/ml) | Tmax (h) |
| 10 | 14 | 102 | 7.5 |
| 11 | 9 | 113 | 7 |
| 11B | 11 | 117 | 7 |
| 32 | 11 | 106 | 10 |
| 33 | 12 | 100 | 16 |
| 35 | 11 | 94 | 10 |
| 36 | 11 | 123 | 10 |
| 38 | 11 | 72 | 12 |
| 40 | 12 | 108 | 9 |

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A sustained release pharmaceutical composition comprising the compound (+)-(2S,3S)-2-(3-chlorophenyl)-3,5,5-trimethyl-2-morpholinol or a pharmaceutically acceptable salt or solvate thereof.

2. The sustained release pharmaceutical composition for oral administration to a human subject comprising the compound of claim 1 or a pharmaceutically acceptable salt or solvate thereof.

3. The sustained release pharmaceutical composition comprising the compound of claim 1 or a pharmaceutically acceptable salt or solvate thereof, which composition is adapted to provide an effective therapeutic dosage of said compound to a human subject by means of once-daily oral administration.

4. The sustained release pharmaceutical composition comprising the compound of claim 1 or a pharmaceutically acceptable salt or solvate thereof, which composition is adapted to provide an effective therapeutic dosage of said compound to a human subject by means of once-daily oral administration.

5. The sustained release pharmaceutical composition as claimed in claim 1 in unit dose form wherein the unit dose contains from 100 mg to 160 mg, expressed as the weight of the free base, of the compound of claim 1 or a pharmaceutically acceptable salt or solvate thereof.

6. The sustained release pharmaceutical composition as claimed in claim 1 which comprises at least one acidic stabiliser.

7. The sustained release pharmaceutical composition as claimed in claim 1 wherein in a phosphate buffer, (pH 6.83, in USPII apparatus at the discriminating paddle speed of about 50 rpm, between about 15% and about 35% of the compound of claim 1 is dissolved within 1 hour; between about 35% to about 65% of said compound is dissolved within 4 hours; and not less than about 55% of said compound is dissolved within 8 hours.

8. The sustained release pharmaceutical composition as claimed in claim 1 in the form of a matrix tablet, the core of which tablet comprises one or more rate-controlling polymers, one or more fillers, and at least one acidic stabiliser.

9. The sustained release pharmaceutical composition as claimed in claim 8 wherein the amount of rate-controlling polymer(s) is in the range of about 20% w/w to about 50% w/w.

10. The sustained release pharmaceutical composition as claimed in claim 8 wherein the rate-controlling polymer is hydroxypropylmethylcellulose.

11. The sustained release pharmaceutical composition as claimed in claim 8 wherein the amount of filler(s) is in the range of about 15% wow to about 70% w/w.

12. The sustained release pharmaceutical composition as claimed in claim 8 wherein the filler is microcrystalline cellulose.

13. The sustained release pharmaceutical composition as claimed in claim 8 wherein sodium bisulphate is included as an acidic stabiliser.

14. The sustained release pharmaceutical composition as claimed in claim 13 wherein the amount of sodium bisulphate is in the range of about 0.05% to about 2% w/w.

15. The sustained release pharmaceutical composition as claimed in claim 8 wherein the tablet is film-coated.

16.-18. (canceled)

19. A method of treatment of a disorder or a condition in a human subject selected from depression, pain, chronic fatigue, obesity, anxiety disorders and mixed depressive-anxiety disorder comprising oral administration to said subject of a sustained release pharmaceutical composition as claimed in claim 1.
